(19) United States
(12) Patent Application Publication
(10) Pub. No.: US 2007/0179626 A1
(43) Pub. Date: Aug. 2, 2007

(54) Functional joint arthroplasty method

(76) Inventors: Jose Luis Moctezuma de la Barrera, Freiburg (DE); Stuart Lee Axelson JR., Succasunna, NJ (US); Peter Zimmerman, Freiburg (DE)

Correspondence Address: MCCRACKEN & FRANK LLP, 200 W. ADAMS STREET, SUITE 2150, CHICAGO, IL, 60606, US

(21) Appl. No.: 11/290,039

(22) Filed: Nov. 30, 2005

(51) Int. Cl. A61F 2/38; A61B 17/58; A61B 17/60; A61B 5/103

(52) U.S. Cl. 623/20.14; 606/90; 606/102; 600/587

(57) ABSTRACT

A method for performing arthroplasty on a joint using a surgical navigation system, the method includes the steps of locating articular anatomical structures using the surgical navigation system; determining biomechanical properties of the joint; and evaluating the soft tissue envelope properties for the joint. The method also includes the steps of displaying an interactive view of the joint, the soft tissue envelope properties, the biomechanical properties and a chosen implant to enable a surgeon to manipulate simultaneously the soft tissue envelope properties, the biomechanical properties and the chosen implant on the interactive view; preparing the joint to receive the chosen implants; and installing the implants in the prepared joint.

### CROSS REFERENCE TO RELATED APPLICATIONS

Not applicable

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

This invention relates to methods, software and systems to assist in the performance of reconstruction and total and partial replacement surgery for joints. More particularly this invention relates to methods, and software to assist in surgical reconstructive interventions of joints that have kinematic behavior influenced by the soft tissue apparatus surrounding it. Typical examples are surgical procedures of the knee or ankle, such as ACL repair, uni-compartmental or multi-compartmental replacement of the joint surfaces, revision surgery and the like.

2. Description of the Background of the Invention

Current joint reconstruction and replacement surgery, including ankle, knee, shoulder, or elbow arthroplasty is based in large part on standard methods and guidelines for acceptable performance. In this regard, the positioning of the implants into the joint is based on standard values for orientation relative to the biomechanical axes, such as varus/valgus, or flexion/extension, and range of motion. One surgical goal might be that the artificial components used to achieve the reconstruction of the joint should have a certain alignment relative to the load axes. These standards are based on static load analysis and therefore may not be appropriate to establish optimal joint functionality taking into account life style patterns of the individual undergoing surgery. There have been systems that look at the ipsilateral side to gage parameters for the operative joint. Also, there have been kinematic approaches that attempt to determine appropriate values for varus/valgus, flexion/extension, and range of motion. One reason for the need to properly balance unconstrained joints, like the knee, ankle and elbow, is that these joints are held together by the soft tissue, including the ligaments, that surrounds the joint The proper functioning of the joint is dependent on a combination of the proper resection of the joint to receive the implant, the proper choice of implant sizing and the proper balance of the soft tissue relative to the implants and the resection. Currently, this balancing is done by the surgeon based on experience and rule of thumb guidelines.

A computer assisted surgical navigation system normally requires a time consuming setup and registration of the patient's anatomy with either a pre-operative scan or with a three dimensional model that is constructed from reference points obtained from the patient's anatomy. Further, prior computer assisted navigation systems have not assisted the surgeon by providing step-by-step procedures to guide the surgeon in making the proper balance between bone cuts, implant size, and soft tissue constraints or balancing. The necessity of additional steps without corresponding added benefits have kept surgeons from using surgical navigation systems for orthopedic surgeries even though the increased accuracy of the surgical navigation systems could improve the end result for the patient.

### SUMMARY OF THE INVENTION

One embodiment of the present comprises a method for performing arthroplasty on a joint using a surgical navigation system. The method includes the steps of locating articular anatomical structures using the surgical navigation system; determining biomechanical properties of the joint; and evaluating the soft tissue envelope properties for the joint. The method also includes the steps of displaying an interactive view of the joint, the soft tissue envelope properties, the biomechanical properties and a chosen implant to enable a surgeon to manipulate simultaneously the soft tissue envelope properties, the biomechanical properties and the chosen implant on the interactive view; preparing the joint to receive the chosen implants; and installing the implants in the prepared joint.

### BRIEF DESCRIPTION OF THE DRAWINGS

FIG. 1 is a schematic view of a patient's knee that has been prepared for knee replacement surgery using components of one embodiment of a surgical navigation system;

FIG. 2 is a flow diagram of one embodiment of the present invention;

FIG. 3 is a screen shot of a further embodiment showing surveying the biomechanical properties of the knee prior to incisions to open the knee;

FIG. 4 is a screen shot of a still further embodiment showing surveying the biomechanical properties of the knee after the initial incisions opening the knee have been made;

FIG. 5 is a screen shot showing an additional embodiment displaying the calculation of the internal/external axes by different methods;

FIG. 6 is a screen shot of the determination of the natural joint line of a knee made during the open knee survey;

FIG. 7 is a screen shot showing a relationship between varus/valgus and flexion over a range of flexion;

FIG. 8 is a screen shot of one embodiment of balancing of the knee joint;

FIG. 9 is a screen shot of one embodiment of initial implant planning;

FIG. 10 is a screen shot similar to FIG. 9 showing a varus load applied to the joint for implant planning;

FIG. 11 is a screen shot of yet another implant balancing embodiment;

FIG. 12 is a similar screen shot to FIG. 11 showing a result using a different set of criteria;

FIG. 13 is a screen shot of a still further embodiment of implant planning;

FIG. 14 is a screen shot showing one embodiment of an interactive iterative display with the knee in extension;

FIG. 15 is a screen shot similar to FIG. 14 of an interactive iterative display with the knee in flexion;

FIG. 16 is a screen shot showing a further embodiment of an interactive iterative display with the knee in extension;

FIG. 17 is a screen shot similar to FIG. 16 of an interactive iterative display with the knee in flexion;

FIG. 18 is a flow diagram of a further embodiment of the present invention;

FIG. 19 is a screen shot showing yet a further embodiment of the present invention;

FIG. 20 is a schematic view of a patent's ankle that has been prepared for ankle replacement surgery using components of a further embodiment of a surgical navigation system;

FIG. 21 is a schematic view of a patent's elbow that has been prepare for elbow replacement surgery using components of a still further embodiment of a surgical navigation system; and

FIG. 22 is an isometric view of one embodiment of an in-situ distraction device useful with the present invention. used to replace or repair any unconstrained joint, such as the ankle, shoulder and the elbow, as illustrated in FIGS. 20 and 21.

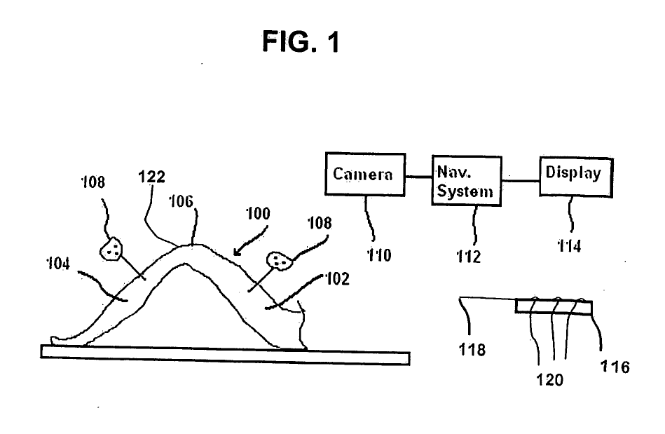

Referring to FIG. 1, a patient's leg 100 is prepared for knee replacement surgery. The leg 100 is bent so that the patient's upper leg or femur 102 is at an angle of approximately 90 degrees to the patent's lower leg or tibia 104. This positioning of the leg 100 places the patient's knee 106 in position for the procedure. Two tracking devices 108 that can communicate with a camera 110 associated with a computer assisted surgical navigation system 112 are associated with the femur 102 and the tibia 104 such that the tracking devices 108 move with the femur 102 and the tibia 104 respectively. The association can be by direct attachment to the bone or by other association methods as discussed hereinafter. The computer assisted surgical navigation system 112 is one that is well known in the art and will not be further discussed here. Suitable surgical navigation systems are described in U.S. Patent Publication No. 2001/0034350, the disclosure of which is incorporated by reference. A typical navigation system 112 will also include a display device 114, such as a computer or video monitor. In addition, most navigation systems 112 will also use specialized tools, such as a pointer 116 that has been previously calibrated to work with the navigation system 112. The calibration of the pointer 116 will enable the navigation system 112 to determine the precise location of a pointer tip 118 by location a series of locator devices 120 such as LED's located on the pointer 116. These locator devices are the same type used for the tracking devices 108.

In addition to the femur 102 and the tibia 104, the knee 106 has a patella 122. The location of the patella 122 can be determined by using the pointer 116. Also because the patella 122 is anatomically tied to the location of the tibia 104, the navigation system 112 can also locate the patella 122 by reference to the location of the tibia 104. Further, because the patella 122 is constrained, it is often only necessary to locate the patella 122 relative to three degrees of freedom. As is well known, the properties of the patella 122 should be considered during knee replacement surgery and any implant that replicates the patella 122 can have an impact on the post surgical functioning of the knee 106.

One relationship that can be considered in certain embodiments is a joint line between the femur 102 and the tibia 104, and the relationship of the femoral-tibial joint line to a joint line between the femur 102 and the patella 122. The joint line is the momentary rotation of the joint in space, in this case the knee 106. The joint line is different than the functional flexion axis. The functional flexion axis describes the overall flexion of the joint. In any joint are typically distinct points of contact between the bones that comprise the joint. Once the surface of one of these bones has been determined, the system can determine the contact points on one bone and link the contact points of the one bone to the contact points of the second bone at the same position of the joint. In the case of the knee, the two contact points on the first bone are related to each other on a line, the joint line. The two contact points on the second bone are also related on the same line initially. However each line can be viewed individually for each of the bones that comprise the joint. Because one or both bones may be deformed as a result of disease or injury, the optimal joint line relative to the reconstructed joint often will be offset from the initial observed joint line for one or both bones.

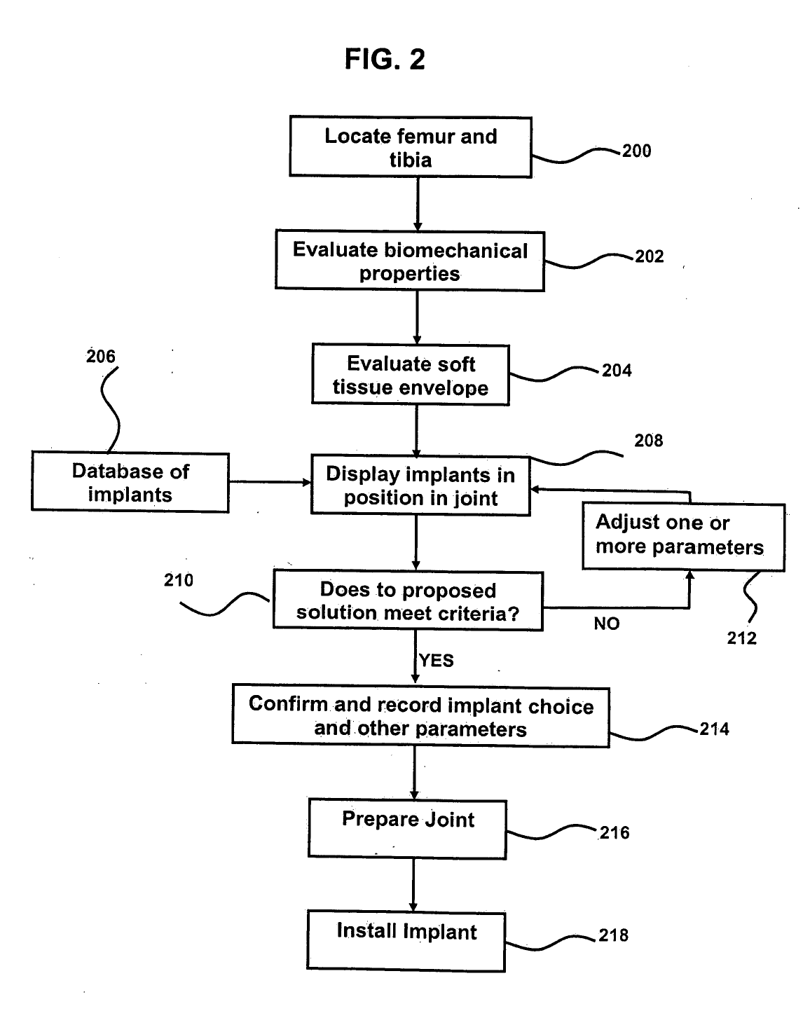

As set out in FIG. 2, a block 200 determines the location of the femur 102 and the tibia 104. As noted above, this is done by associating tracking devices to the femur 102 and the tibia 104 and then manipulating the respective limb within the view of the camera 110 of the navigation system 112. Optionally, the block 200 can also determine the location of the patella 112.

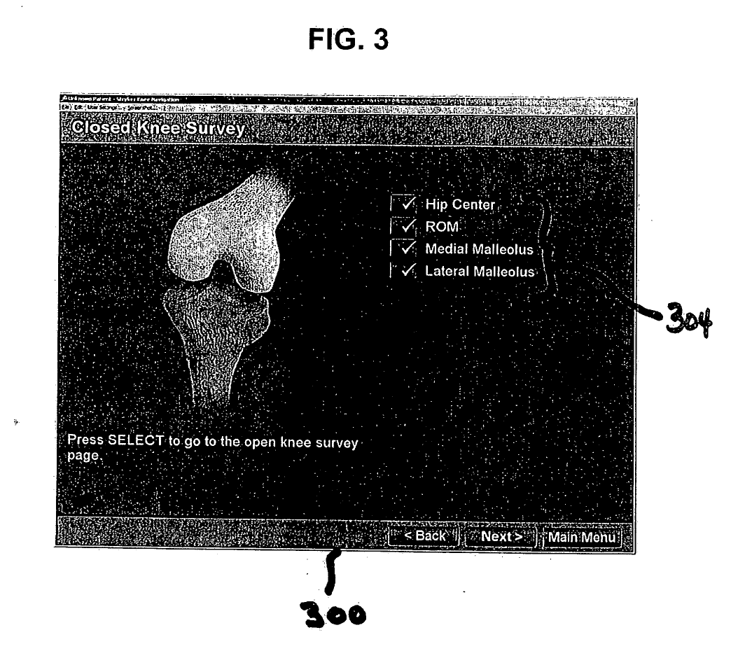

Control then passes to a block 202 where the biomechanical properties for the femur 102, the tibia 104, the knee 106, and optionally the patella 122 are determined. Some of the biomechanical properties can be determined before the actual incision is made. For instance, after the tracking device 108 is attached to or associated with the femur 102, the location of the femoral head can be determined by manipulating the femur 102 to rotate about the hip socket. In addition, an initial functional flexion axis analysis and a range of motion analysis can be performed by manipulating the knee 106 through the entire range of motion of the knee 106. Also, the location of the lateral malleolus and medial malleolus can be determined using the pointer 116 and touching the pointer tip 118 to the appropriate bony landmarks on the ankle. The navigation system 112 can display a series of screens on the display 114 of the type as shown in FIG. 3 to guide the surgeon in performing the closed knee evaluation of the knee 106. The navigation system 112 will track the location of the tracker 108 that is associated with the femur 102 and indicate when sufficient points have been recorded to accurately locate the femoral head and determine the range of motion and the initial functional flexion axis. In addition, the relationship of the patella 122 to the tibia 104 can also be determined at this time. Some of these evaluations on the closed knee 106 can be performed earlier as part of a pre-operative work up and recorded for use during a later surgery.

Traditionally knee replacement has had enormous focus around the tibio-femoral joint. Nevertheless the knee is a tri-compartmental joint with the patella gliding in the femoral trochlear groove transmitting quadriceps forces onto the tibia. Failing to re-establish proper patello-femoral kinematics often yields to unsatisfactory pain levels after implantation. One of the most important parameters to take into consideration in order to re-establish proper operation of the quadriceps mechanism is the distance of the patella to the functional flexion axis. If the distance is increased, so called overstuffing of the patella occurs that leads to a limited range of motion and pain. If the distance is decreased, the efficacy of the quadriceps mechanism is compromised and mal-tracking of the patella may occur. Medial-lateral displacement of the patella is in turn influenced by internal-external rotation of the implant. If there is a gross mismatch of the natural track of the patella and the reconstructed trochlear groove of the implant, dislocation and pain may occur. The rotational degrees of freedom of the patella, namely tilt, rotation and flexion, are coupled to the above parameters and are of secondary importance.

Tracking only the trajectory of the patella simplifies the needed devices by not having to implement all 6 degrees of freedom. This is important because the patella is relatively small structure and the kinematics of the patella can be altered if a foreign object attached to the patella. Furthermore, the patella has a constant relationship to the tibia. It is attached by a tendon to the proximal spine of the tibia where the forces from the quadriceps mechanism are received and passed on to the foot. This relationship is seldom affected and represents a very faithful landmark intrinsically describing some of kinematic parameters of the knee joint. These parameters are very helpful while re-establishing the function of the knee but are especially helpful in cases where no other references may be available as for example in revision surgery where the tibio-femoral joint has been replaced.

After the closed knee biomechanical data has been determined and recorded, the initial incision is made to the knee 106 to expose the distal portion of the femur 102, the patella, and the proximal portion of the tibia 104. The surgeon can then determine other landmarks using the pointer 116 as guided by the navigation system 112 through the various screens displayed on display 114. Typical landmarks include the surface of the lateral and medial condyles, the tibial plateau dishes, and other landmarks.

Certain landmarks can be inferred from the digitization of other landmarks. For instance the surfaces of the femoral condyles can be determined by assessing the location of the surface of the tibial plateau throughout the range of motion. Because of the envelope constraints on the knee joint, the surface of the femoral condyles will sweep the tibial plateau as the knee joint is flexed throughout the range of motion and can be computed once these two properties or landmark locations are known. While there can be a lift off situation, this still does not have a significant impact on the computation because the femur can not penetrate into the tibia. Also, the center of the knee can be computed as the most distant point of the groove from the center of the femoral head.

In addition through further geometrical analysis of the obtained surfaces fundamental properties can be derived, such as radiuses of curvature or axes of rotation. For example in a knee 106 the internal/external rotation of the femur 102 can be computed by a cone or cylinder fitted to the geometry of the posterior condyles, which are usually unaffected by the disease condition of the knee 106. The femoral-tibial joint line is constructed by joining the two points of contact of the tibial dishes with the femoral condyles. Because one of the condyles is normally eroded, due to the disease condition, the joint line is affected and reflects the varus or valgus deformation. The varus/valgus deformation has been determined during the closed knee range of motion analysis and the surgical navigation system 112 can compute the current joint line and also the system can propose a restored joint line that will reflect the knee in a repaired state. The restored joint line can be used as a target by the surgeon during the balancing of the knee soft tissue, the implants and the modifications made to the bones to receive the implants.

Further to the geometrical analysis of the pairing surfaces of a joint, its functional kinematic data can be analyzed to derive momentary or instantaneous axes of rotation or overall axes of rotation by analyzing portions or all of the instantaneous axes. One of the best known and most widely used techniques is the helical axis computation which is said to describe the home-screw mechanism of unconstrained joints. In the case of the knee, the flexion axis of the patello-femoral joint as well as the tibio-femoral joint can be computed by passively moving the joint throughout its range of motion. The so derived flexion axis of a diseased joint will undoubtedly reflect the diseased kinematics of the joint. Partial correction of some of its degrees of freedom will be necessary before it is used as a guide for the surgical measure. The corrections may be derived by combining information or constraints given by the biomechanical axes of the joint and/or by dynamic load transfer patterns while ranging the joint. Another alternative is by combination of information provided by unaffected degrees of freedom of other pairing surfaces of the joint. In the case of the knee 106, a possible combination could be perpendicularity of the derived functional flexion axis to the biomechanical axis, translational constraints given by the patello-femoral flexion axis and its internal/external rotation dictated by the conical fit axis of the posterior condyles of the femur.

Another method to re-establish normal kinematics of the affected joint is to assess the kinematics of the non-affected ipsilateral side. These parameters can be extracted by the same methods and are expressed preferably in terms of local non-affected anatomical structures to enable its transfer to the affected site after identification of the corresponding anatomical structures. In the case of the knee 106, a local reference can be established by the intercondylar notch and the anterior cortex of the femur 102. The identification of these structures can be done intra- but preferably preoperatively with any non-invasive imaging technology. In the case where the functional analysis is not done with the same modality as the one used for the identification of the reference structures the registration of both modalities is necessary. A preferred embodiment uses ultrasound as imaging modality. This coupled with tracking technology relates kinematic information to the underlying reference structures and minimally invasive tracking devices as described in published U.S. Patent application No. 2005/199,250, published Sep. 15, 2005, the disclosure of which is hereby incorporated by reference.

The computed kinematics information in form of restored functional axes and joint lines of the joint surfaces may not only be used as a guide for driving the position of prosthetic components but also for establishing an optimum between the kinematics constraints of the individual's joint and the prosthetic system being used. In a scenario where multiple prosthetic systems or surgical techniques are available the computer system may choose the optimal implant and propose an optimized position to best fit constraints given by the function of joint and those of the implant. A further example of an optimization criterion could be optimal performance for a given activity of the individual that best restore his or her quality of life.

A block 204 evaluates the soft tissue surrounding the joint. The soft tissue can be evaluated by further manipulating the knee 106 and also by the use of strain gauges or similar devices. The knee includes four main ligaments that interact with the tibia 104 and the femur 102 to form a stable knee 106. The tension on these ligaments must be properly balanced to provide stability to the knee 106. The surgical navigation system 112 can also guide the surgeon through the soft tissue analysis and based on the particular manipulation that is performed record values for the tension of the various ligaments and muscles of the knee 106. Other methods of acquiring the soft tissue tension values can also be used.

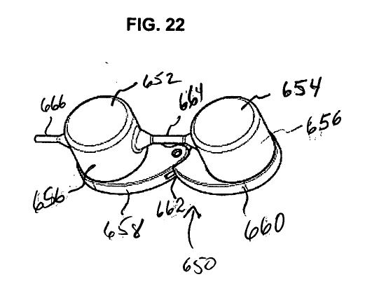

Particularly useful are the in-situ devices of the type shown in FIG. 22 and described hereafter that do not require the modification of the soft tissue joint envelope, such as everting the patella in the case of the knee. Such devices can be balloon systems that can exert distraction forces that simulate normal activity and thus providing a close approximation to the forces experienced during voluntary motion of the joint. These systems require little or no preparation of the site in order to be used and can be introduced through small incisions or through a cannula system such as those used in arthroscopy. Furthermore these systems allow the assessment of function of the joint throughout its entire range of motion as opposed to typically representative stances in flexion and extension. In addition, other devices than those shown in the application can also be used. For instance, the devices shown in U.S. Pat. Nos. 6,702,821, and 6,770,078, the disclosures of which are hereby incorporated by reference, can be used as well.

Another benefit of in-situ devices is the ability to iteratively establish or capture the functional parameters, as the functional flexion axis of the joint that exactly describes the actual state of the soft tissue envelope of the joint. Through instant assessment of the effect of a given soft tissue measure are important to precisely drive the desired soft tissue correction.

The usage of more sophisticated distraction devices in which force or pressure sensing elements have been incorporated can yield precise information on loading pattern characteristics for the joint throughout range of motion. These in turn can be used to establish a certain soft tissue management strategy in which specific group or bundles of bands are selectively targeted to affect the load or force pattern at a specific flexion or kinematic state of the joint. These devices can transmit wirelessly in a real time fashion the information to the computer system for on the fly analysis. The information can then be displayed numerically or graphically in relationship to the established model of the joint. Using this information the computer system can also deliver the most likely soft tissue management strategy based on e.g. an underlying expert system.

The surgical navigation system 112 will also include a database 206 of implant components in digitized form. A block 208 takes the values from the location analysis 200 of the femur 102 and the tibia 104, the biomechanical properties analysis 202, the soft tissue evaluation 204, and the database 206. Using all these values, as well as other criteria including but not limited to gender, age, race, life style, and the like, the block 208 simultaneously solves for the functional goal and displays the calculated result, including a suggested implant from the database of implants 206, on an interactive screen on the display 114. One possible element of the functional goal in one embodiment of the present invention can be the restored joint line. This can be shown on the interactive screen along with other values relative to restoring the joint. Control passes to a block 210 that enables the surgeon to manually adjust the chosen functional goal and other values if necessary to reflect the surgeon's experience with the procedure. If the surgeon determines that the solution shown by the block 208 is not optimum, control will pass via a NO branch to a block 212 that allows the surgeon to digitally manipulate the joint and possibly change the suggested implant or other parameters as shown on the interactive screen. After the changes are made, the navigation system 112 will recalculate the result and the block 208 will display the updated result. If at this point, the surgeon believes that the proposed solution meets the surgical objective, then control will pass via a YES branch to a block 214 that asks for confirmation and recording of the choice of implant and other parameters. At this point, the surgeon in a block 216 will prepare the joint to match the chosen solution. The navigation system 112 can guide the surgeon through the procedure and make suggestions of modifications necessary to achieve the desired outcome or the surgeon can proceed in a conventional fashion to prepare the joint without the navigation system 112. After the joint is prepared in the block 216, the implant is installed in a block 218. Again, if the surgeon chooses, the navigation system 112 can guide the surgeon through this procedure as well. As will be discussed later, the installing of the implants can also include the use of trial implants that replicate the final implants and allow the surgeon to test the configuration of the joint before the final implants are permanently placed in the joint.

The preparation of the joint according to the established goal can be performed manually with the aid of navigation but also with any type of passive, semi-active or active envelope constraining devices, with master-slave manipulators or with autonomous in-situ mounted or external manipulators, including telemanipulators.

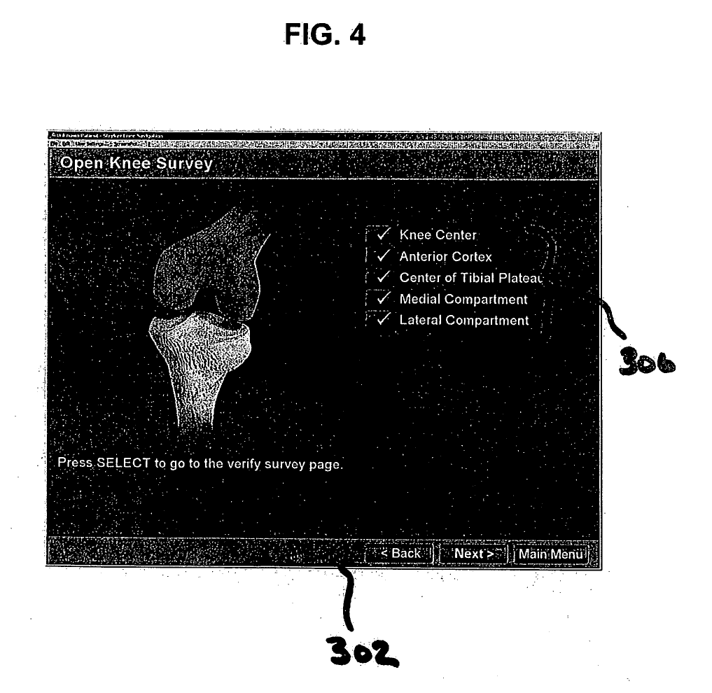

FIGS. 3 and 4 are two screen shots 300 and 302 that show the surveying of the knee joint set out in the block 202. As shown, the display 114 will guide the surgeon through the steps needed to survey the knee joint in preparation for the procedure. It also provides checklists 304 and 306 of the parameters that should be determined and the navigation system 112 will also provide additional screens to assist the surgeon in determining the location of the hip center, the range of motion of the knee, the location of the medial malleolus, and the lateral malleolus. In addition, after the knee joint has been opened, the pointer 116 can be used along with the navigation system 112 to determine the knee center, anterior cortex, the center of the tibial plateau, the medial compartment and the lateral compartment. The navigation system 112 can guide the surgeon through the location of these landmarks or just record the location when the surgeon makes a manual location of the landmark using the pointer 116.

Other biomechanical properties and landmarks can be determined by the navigation system 112 by indirect digitization using combinations of the above determined landmarks as noted above. The surface of the femoral condyles can be determined by combining the range of motion analysis with digital location of the tibial plateau.

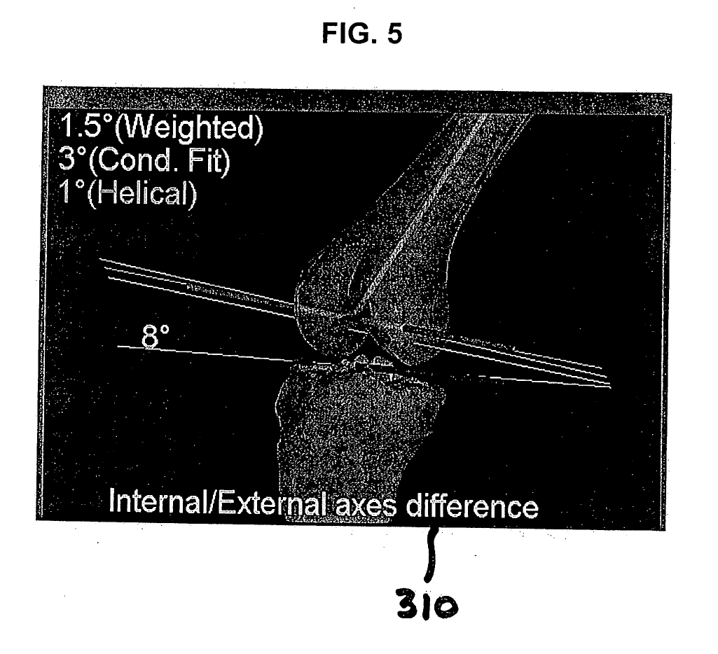

The internal/external rotation of the femur 102 can also be determined by a variety of methods. For instance the internal/external rotation can be derived from the early, 0° to 45°, flexion. There are a number of well known algorithms that can make this calculation including helical axis, residual minimization and other similar geometrical optimization techniques. Alternatively the internal/external rotation can be derived from the shape of the posterior condyles. Normally the shape of the condyles in deep flexion, greater than 90°, are unaffected by a possible disease condition of the joint. The internal/external rotation is determined by fitting a cone or cylinder to the femur 102 as the knee 106 is flexed relative to the femur 102. FIG. 5 shows a screen shot 310 that displays the results of the location of the internal/external axes by two different methods as described above and also by an averaging or other weighting of the two results of the internal/external axis of rotation. The tibial internal/external axis of rotation can also be determined in a similar fashion.

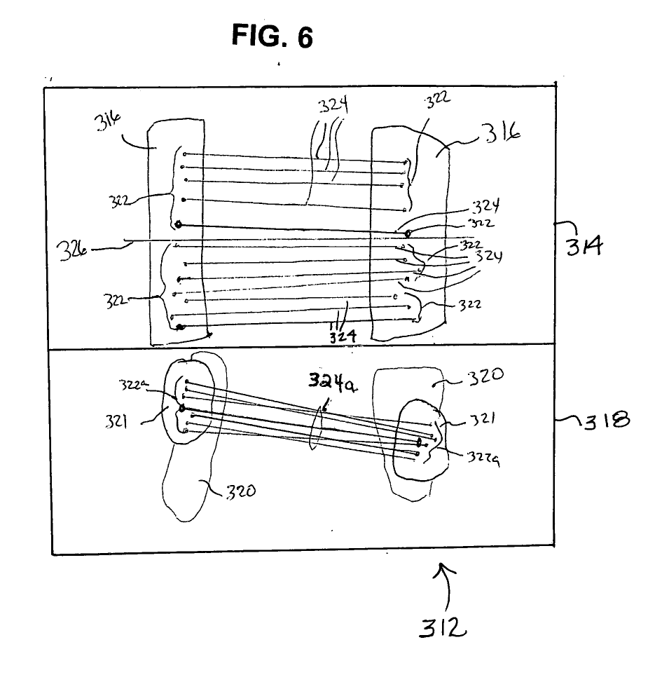

FIG. 6 shows a screen shot 312 of the determination of the initial joint line. In an upper pane 314, a representation of the femoral condyles 316 is shown and in a lower pane 318 is shown a representation of the tibial compartments 320. A pressure zone 321 is where the femoral condyles 316 contact the tibial compartments 320. A series of contact points 322 represent points of contact on each of the femoral condyles and a similar series of contact points 322a represent the points of contact on the tibial compartments. A series of lines 324 are momentary axis of rotation joining pairs of contact points 322 of the femoral condyles 316 with the tibial compartment 320. For every joint line 324 on the femoral condyles 316 there is a corresponding joint line 324a. A functional flexion axis 326 for the knee 106 is also shown for reference. A restored joint line can be calculated by the system and used as a surgical goal of the surgeon and the navigation system 112. The restored joint line is determined by taking the most prominent point on the femoral condyles 316 and taking a line that intersects this most prominent point on the femoral condyles 316 that is perpendicular to the mechanical axis of the femur 102.

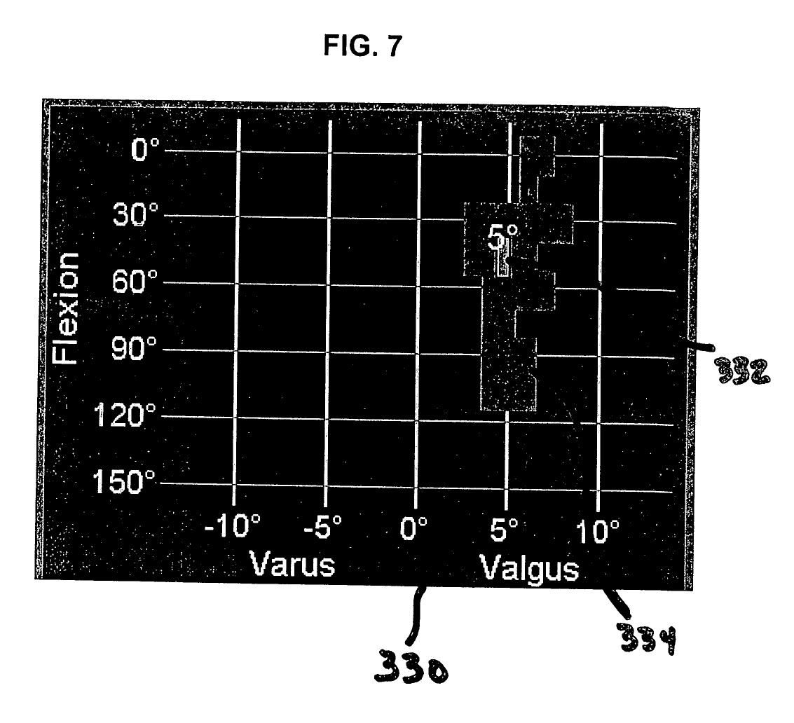

In the block 204, the soft tissue envelope is evaluated. One method of conducting this evaluation is to flex the open knee joint throughout the range of motion while at the same time applying a varus or valgus load to the knee joint. The surgeon will manipulate the knee joint by flexing the knee and press on either the lateral surface to apply a varus load or the medial surface to apply a valgus load. FIG. 7 is a screen shot 330 plotting loads against the flexion angles. A cursor 332 at 5° valgus and 45° is the current position of the joint. An area 334 in the plot shows the extent of laxity of the joint at particular flexion angles. The surgeon is interested in seeing the amount of laxity of the soft tissue that constrains the knee joint 106. The amount of laxity will have an impact on the selection of the possible implant as well as the location of the bone cuts that the surgeon will need to make to prepare the joint to receive the implants. Also, this evaluation will suggest to the surgeon the type and amount of any soft tissue releases that will be necessary to balance the knee 106 after the implants are in position. In addition, it will also be possible to determine similar information relative to the restored joint line.

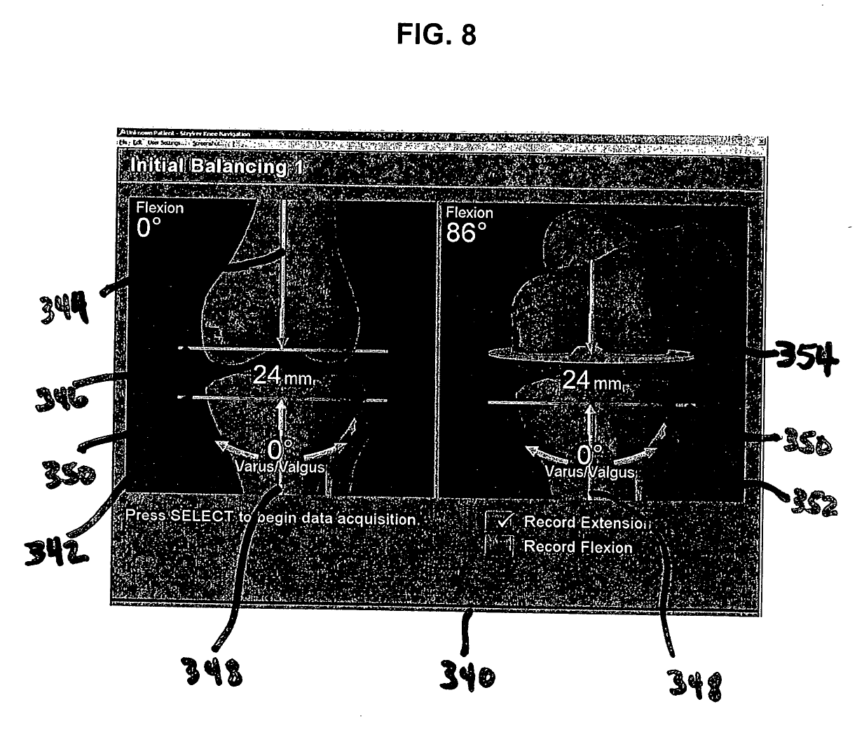

As an initial aspect of the step of the block 208, a screen shot 340 similar to FIG. 8 will assist the surgeon in the initial balancing of the joint and the initial determination of the location of the cuts that will need to be made to the tibia and to the femur. For most reconstruction surgeries, the amount of varus and valgus deflection will be as close to zero as possible. In a left pane 342, the knee 106 is shown at 0° flexion. A line 344 is the mechanical axis of the femur. This shows the case where a femoral cut line 346 will be perpendicular to the mechanical axis of the femur. A line 348 shows the mechanical axis of the tibia and a tibial cut line 350 shows the proposed location of the cut to the tibial plateau. This will result in a gap of 24 mm as shown in FIG. 8. A right pane 352 shows the joint at 86° flexion. A line 354 shows the cut that will be made to the posterior portion of the femur. This also will provide a 24 mm gap. Typically, the cut lines 346 and 354 will be parallel to each other. This provides an easier solution to the particular implants to be chosen from the database 206. Also, the gap should be similar during all degrees of flexion of the joint so that the final implants will function as smoothly as possible when the patient is walking and engaging in normal activities appropriate to the patient's life style after the procedure.

The analysis of the soft tissue can be done by manipulation of the knee 106 or it can alternatively be done by making a perpendicular cut to the tibial plateau to provide space for an in-situ, patella in place balancer device. The device can be of any suitable construction so long as the device will enable the surgeon to tense the knee joint 106 against the soft tissue. One suitable balancing device is shown in FIG. 22 that will be described in more detail below. Because the femur will ride over the balancer device, it is not necessary to make any cuts to the femur at this time. The surgeon can flex the knee joint 106 over the entire range of motion and can establish a functional flexion axis of the knee joint 106. This provides a time savings as well as adding flexibility to the procedure. The surgeon can also determine the placement of the femoral cuts after the soft tissue has been evaluated. The surgeon will have greater control over the choice of implants and be able to minimize, if desired, the amount of change needed to the soft tissue. Depending on the training of the surgeon, the surgeon may want to respect the soft tissue and make as few changes to the soft tissue as possible. An alternative training is to make more changes to the soft tissue than to the boney structure. There are also schools of thought that provide a combination of the above two approaches. Depending on the approach, the surgeon is in control and can determine the amount of change to the soft tissue or to the bone.

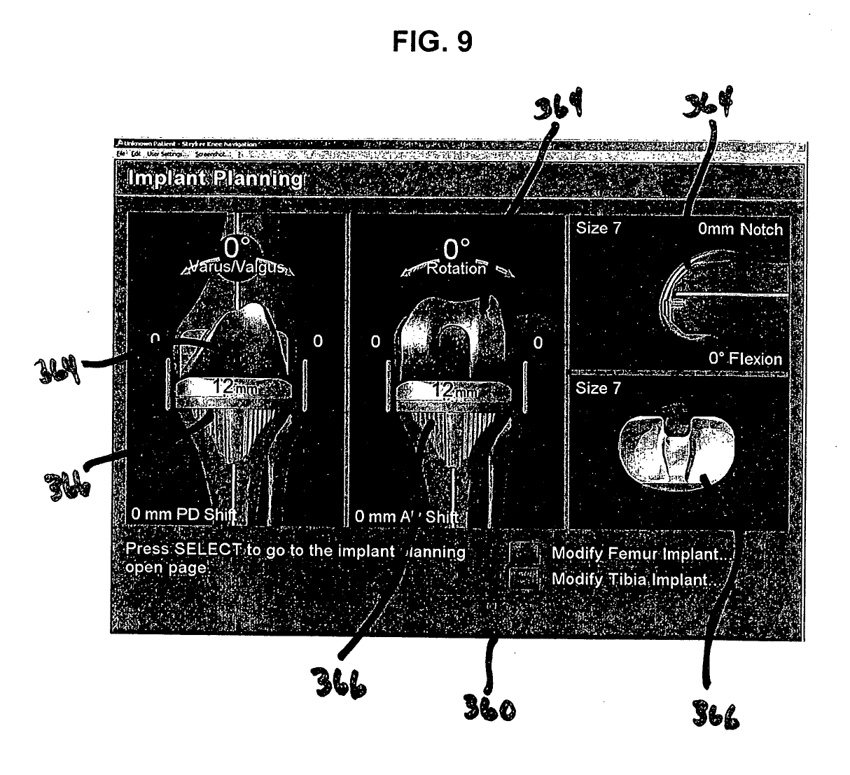

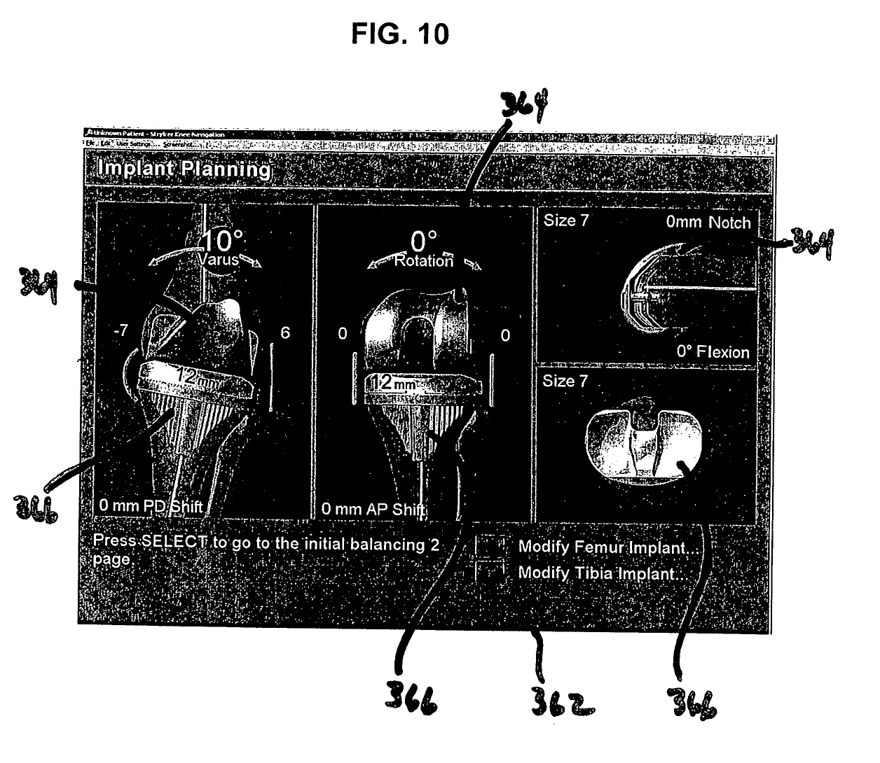

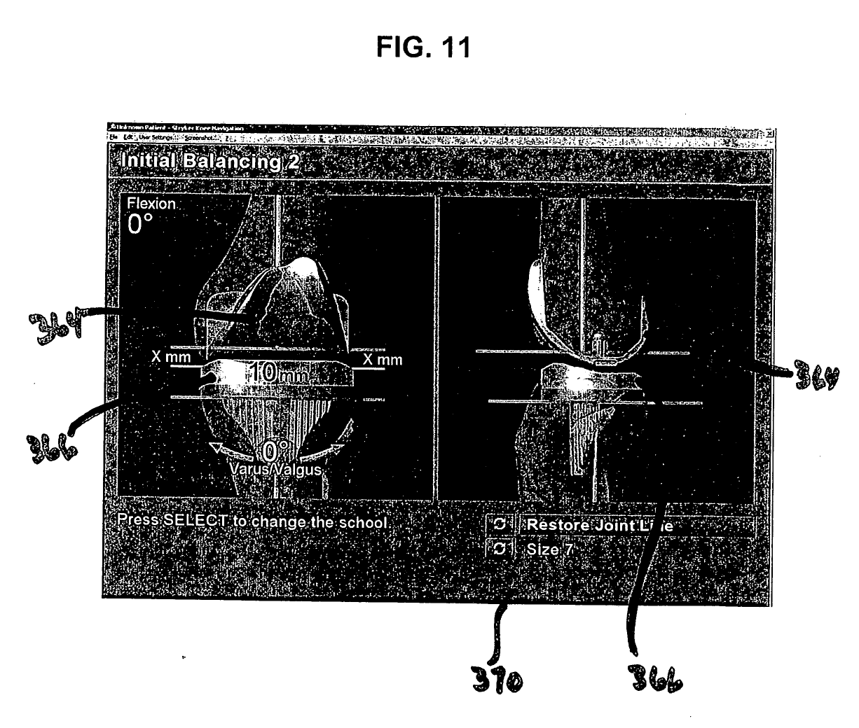

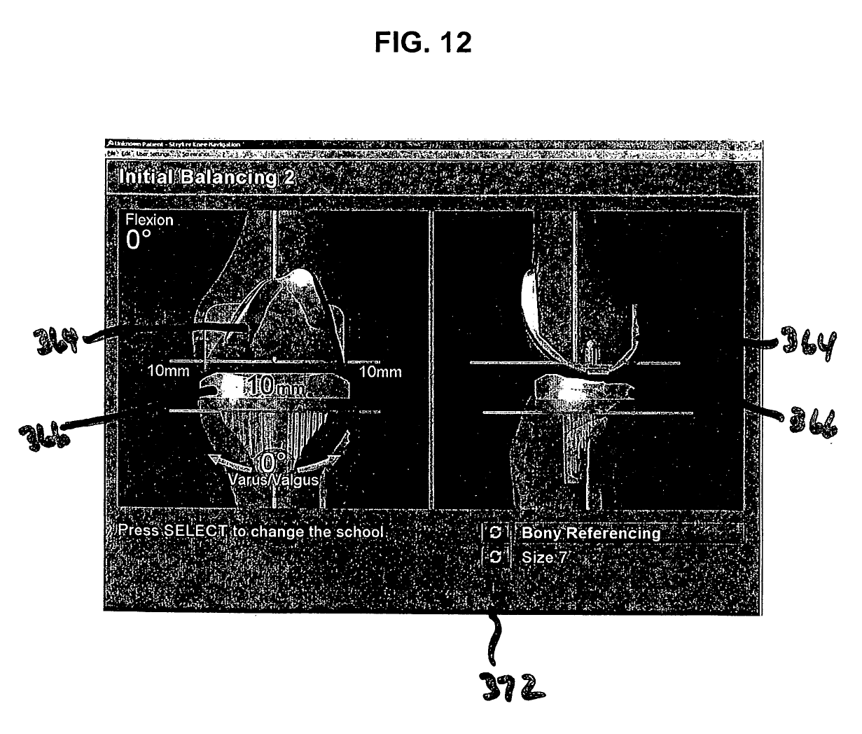

FIGS. 9, and 10, show two screen shots 360 and 362 that have implants 364 and 366 virtually placed in the knee joint 106. The particular implants 364 and 366 are chosen based on the parameters of the knee joint 106 when combined with the properties of the various implants in the database 206. FIG. 9 shows the knee joint 106 without any side load or pressure. FIG. 10 shows the same knee joint 106 subjected to a 10° varus loading. This shows the ability of the surgeon to modify the choices made prior to committing to a particular joint configuration. The surgeon can also subject the knee 106 to virtual forces as shown in FIG. 10 and observe the effect of these forces on the knee joint 106 and the proposed implants 364 and 366. FIGS. 11 and 12 show screen shots 370 and 372 of an alternative embodiment similar to FIGS. 9 and 10. FIG. 11 shows the proposed implants 364 366 that have been selected to restore the joint line. FIG. 12 shows the proposed implants 364 and 366 that will achieve the boney referencing goal.

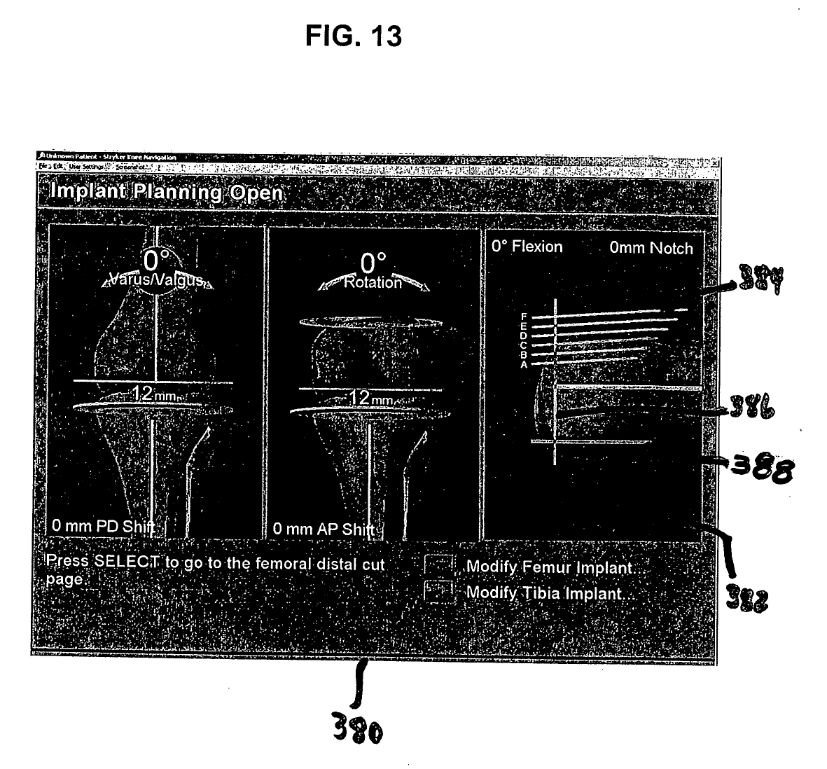

FIG. 13 shows a screen shot 380 of an alternative embodiment what that does not use a database of components or provides an option for the surgeon when there is no good solution for the implant from the database. In a right pane 382, the femur 102 is shown with a sizing grid 384. This shows the placement of various implants relative to a distal 386, an anterior 384, and a posterior 388 portion of the femur 102. After the surgeon manually chooses an implant, the navigation system 112 can display the chosen implant in place in the joint as above. The surgeon then can manipulate the knee 106 as above.

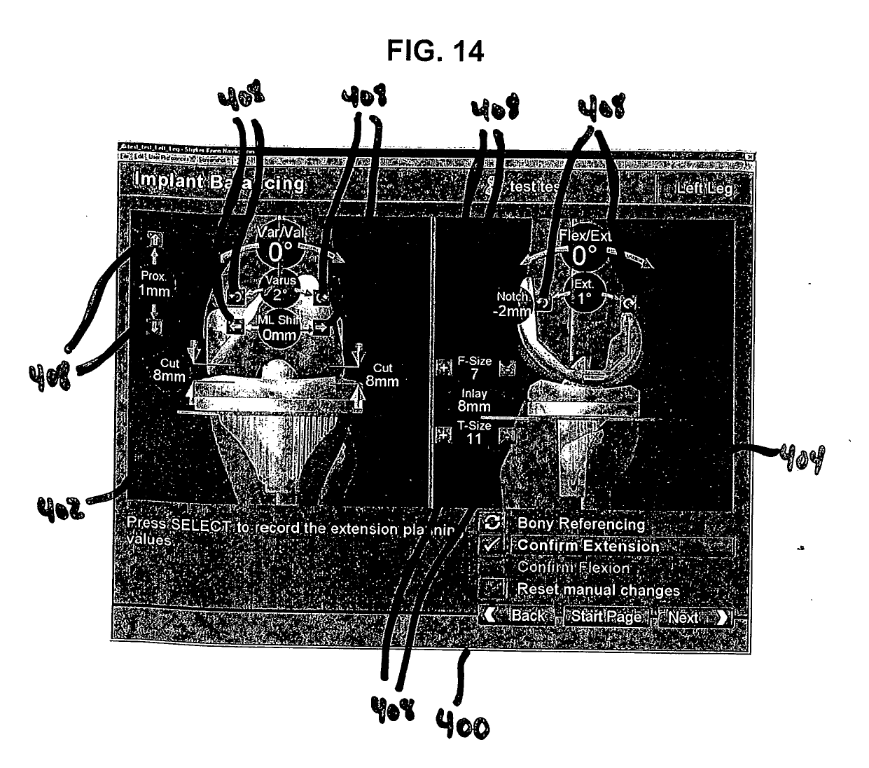

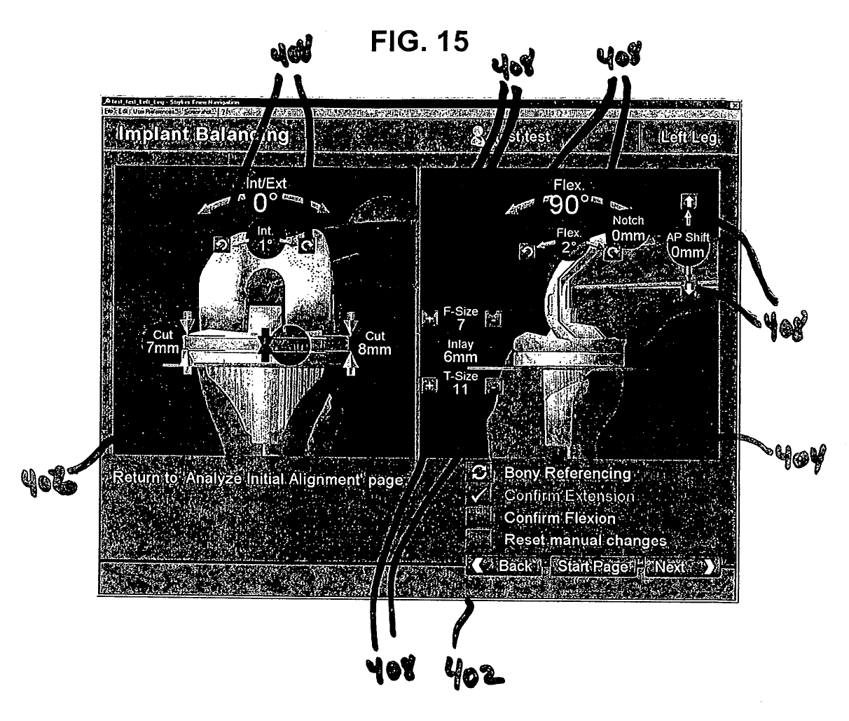

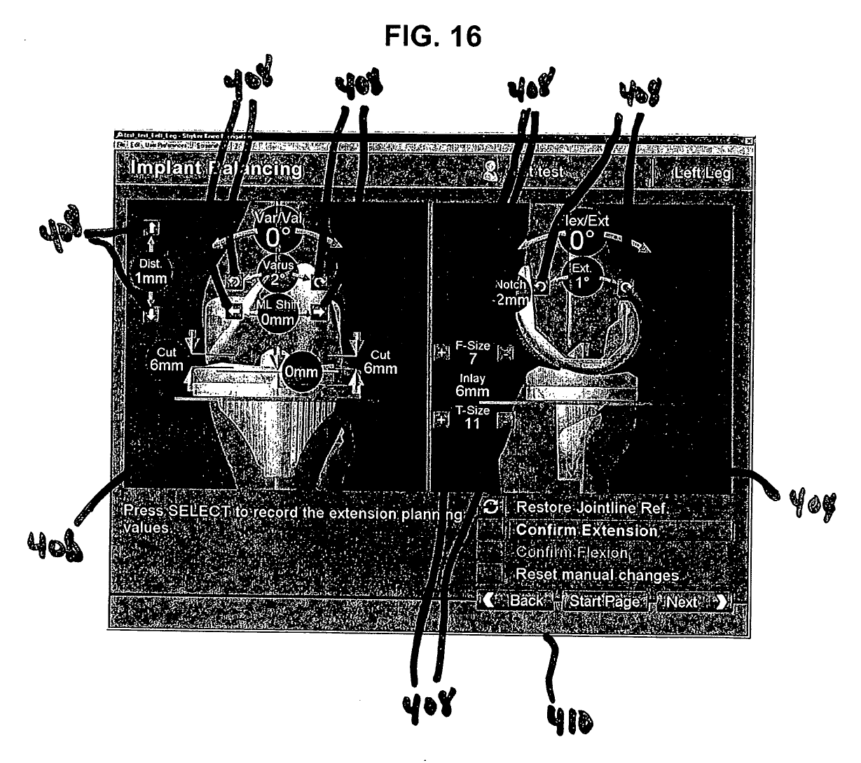

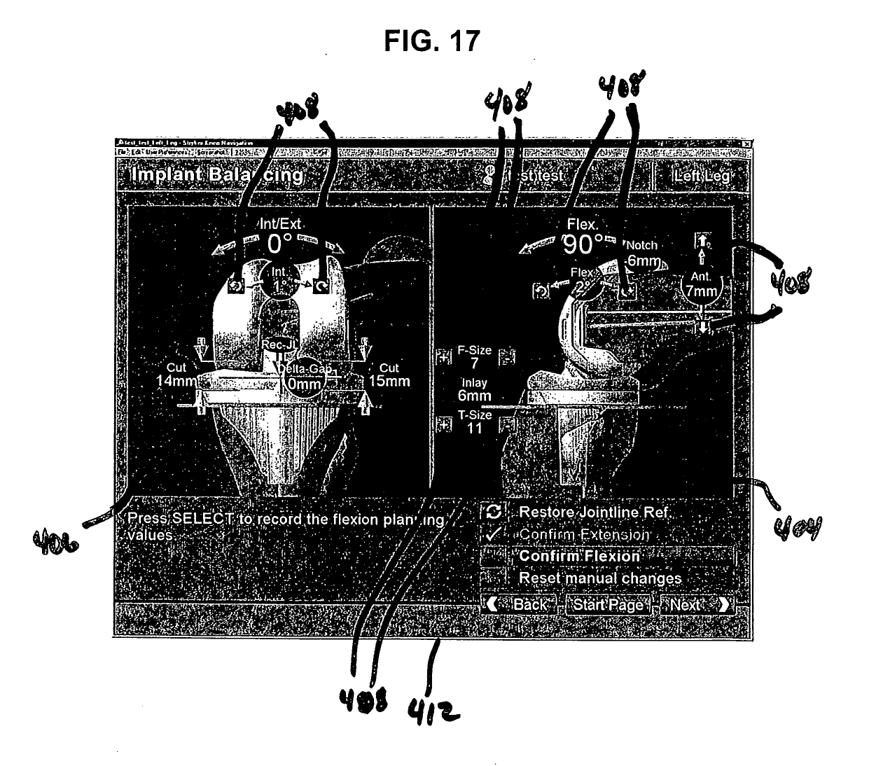

FIGS. 14 and 15 show screen shots 400 and 402. In this embodiment, the balancing is done relative to bony referencing. Both panes 404 and 406 have a series of buttons 408 that can virtually vary any of the values shown. The result of the variation of the value will be immediately shown in both panes 404 and 406. This will provide the surgeon with greater control over the choices that the navigation system 112 may suggest. In a similar manner, FIGS. 16 and 17 show screen shots 410 and 412 of a still further embodiment that does the balancing by respecting the restored joint line.

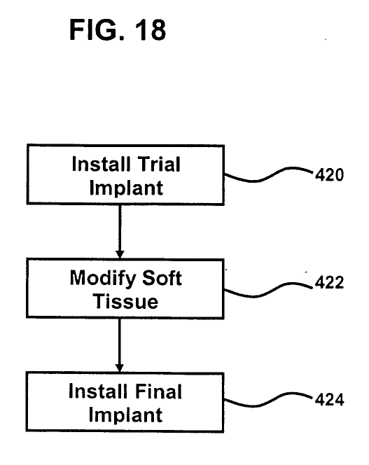

As shown in FIG. 18, the installation of the implants can include multiple steps. In a block 420, the navigation system 112 assists the surgeon in the placement of trial implants to the preparation of the joint and the modification to soft tissue tension. It may be necessary in a block 422 to adjust the soft tissue tension by known methods. The navigation system 112 will assist the surgeon by guiding the tools to the proper location for releasing tension of a particular ligament or ligament bundle. When the surgeon is satisfied with the trial implants and the soft tissue, the system proceeds to a block 424 that assists the surgeon in the placement of the final implants.

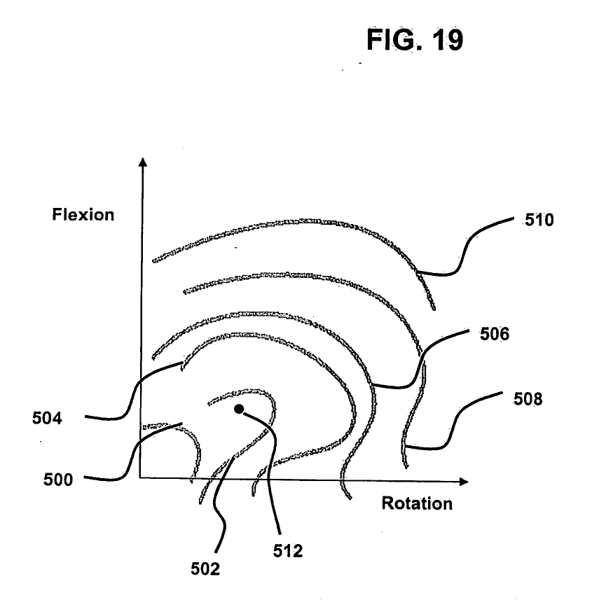

FIG. 19 shows another method of visualizing the interactive nature of the present invention. The diagram shows various curves that are a series of solutions for the optimal positioning of the implant for a particular implant combination. The surgical navigation system 112 displays curves 500 through 510 relative to the flexion and rotation of the knee 106. The point on the display 512 indicates the current position of the knee joint 106 and of the proposed implants. By manipulation of the knee 106, the surgeon can choose the combination of implants and soft tissue corrections that produce a desired result or goal.

Visualization of the above disclosed information as, flexion axis, joint line, alignment, load distribution through out range of motion, implant size and position, etc. is challenging and can be sometimes overwhelming. An effective and above all ergonomic method to convey this complex context is by virtue of augmented reality techniques where through projection techniques the graphical information can be overlaid onto the anatomical structures being addressed. This results in an intuitive context oriented visualization of the required information. For instance the chosen implant can be superimposed at the correct position directly on the anatomical structure giving the surgeon the opportunity to assess the overall fit and required preparation of an intact joint.

The system and methods of the present invention have been described using the knee 106 as an example joint. It should be understood that the knee 106 is the most complicated unconstrained type joint. As such, the methods of the present invention can be applied to other unconstrained joints in the body such as the ankle, shoulder or the elbow. It should also be understood that any type of surgical measure throughout the continuum of care of said joints will profit from the here described methods. The various surgical implants can range from autologous tissue for focal repair to structured load bearing biomaterials to replacement surfaces of inert materials to revision type prosthetic implant. It should also be understood that the methods of the present invention can be accomplished by the surgical navigation system 112 that has software loaded into random-access memory in the form of machine-readable code, such code being executable by an array of logic elements such as a microprocessor or other digital signal processing unit contained within the surgical navigation system 112 or within any standard computer system.

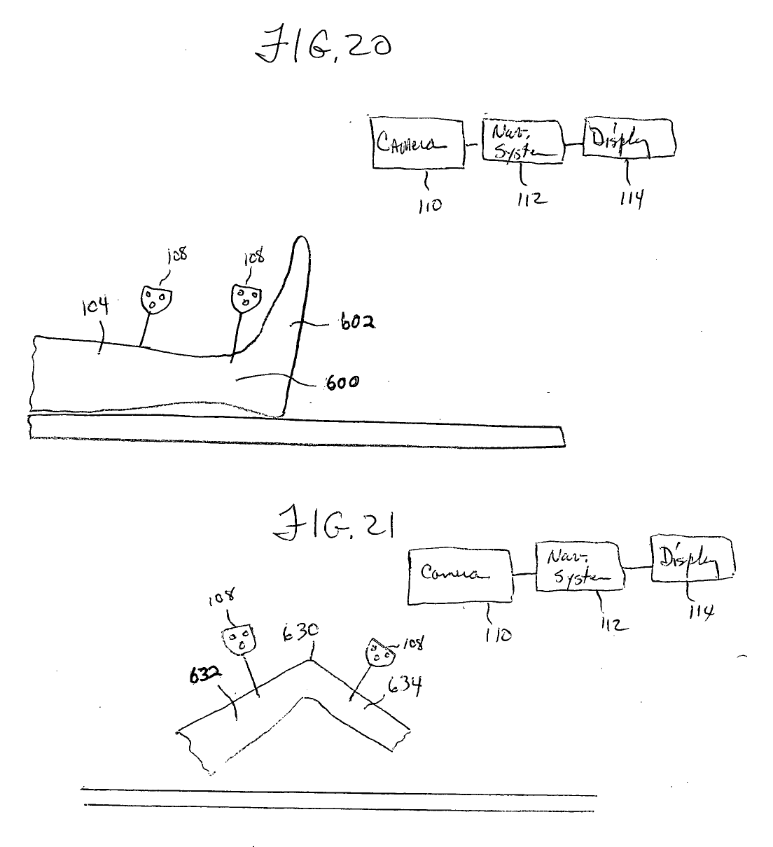

FIG. 20 is a schematic view of an ankle 600 being prepared for ankle replacement surgery. The tracking device 108 is associated with the tibia 104. In addition there is the second tracking devise 108 associated with a talus bone (not shown) of the ankle 600. As the surgeon manipulates a foot 602, the ankle 600 will allow the foot to move up and down. The ankle 600 is comprised of the true ankle joint where the tibia 104, the fibula and the talus come together. Below the talus is the subtalar joint where the talus meets the calcaneus. The true ankle joint allows the foot 602 to move up and down relative to the tibia 104 and the subtalar joint allows the foot 602 to move from side to side. Depending on the procedure, there may also be an optional tracking device 108 associated with the foot 602. Using the same workflow as above for the knee 106, the surgeon can perform the appropriate replacement surgery for the ankle 600. In a similar manner as schematically illustrated in FIG. 21, surgery of an elbow 630 can also be performed. The tracking devices 108 are associated with a humerus 632 and an ulna 634 as shown. Again, replacement surgery of the elbow 630 can be performed using the procedures and workflow outlined above.

FIG. 22 shows one embodiment of a distracting device 650. The device 650 has two expendable bladders 652 and 654. The bladders 652 and 654 can be inflated or deflated to provide pressure for the knee joint 106. In one embodiment the bladders 652 and 654 are separately controllable such and each blades 652 and 654 can exert a different pressure on that compartment of the knee joint 106. In other embodiments, the bladders 652 and 654 exert the same pressure on both compartments of the knee joint 106. The bladders 652 and 654 can be formed from any suitable surgically acceptable flexible material. Examples include plastics as polyethylene terephthalate, polyurethane, polyvinyl chloride, and the like. The bladders 652 and 654 can have a smooth side wall 666 as shown or the side walls 656 can be fluted. The bladders 652 and 654 are mounted on bases 656 and 660 respectively. The bases 658 and 660 can be joined by a hinge 662 or in an alternate embodiment the bases 658 and 660 can be joined so that the bases 658 and 660 cannot move relative to each other. A flexible tube 664 extends between bladders 652 and 654. In our embodiment where the bladders 652 and 654 exert the same pressure, the tube 664 communicates with the internal of the bladder 652. In the embodiment where the bladders 652 and 654 exert different pressure, the tube 664 is in communication with an individual air supply not shown. A second tube 666 connects the bladder 652 with the external air supply. The distracting device 650 can be sized such that the distracting device 650 can be inserted into the knee joint 106 using minimal invasive surgical techniques.

The computer software can be stored in any convenient format usable by computers that can be found within surgical operating rooms. Often, the software will be made available on media such as CD-ROM, DVD-ROM or similar data storage media. In addition, the software can be made available for download though an Internet connection.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

We claim:

1. A method for performing arthroplasty on a joint using a surgical navigation system, the method including the steps of:
locating articular anatomical structures using the surgical navigation system;
determining biomechanical properties of the joint;
evaluating the soft tissue envelope properties for the joint;
displaying an interactive view of the joint, the soft tissue envelope properties, the biomechanical properties and a chosen implant to enable a surgeon to manipulate simultaneously the soft tissue envelope properties, the biomechanical properties and the chosen implant on the interactive view;
preparing the joint to receive the chosen implants; and
installing the implants in the prepared joint.

2. The method of claim 1 where the joint is a knee and the anatomical structures are weight bearing and are the femur, tibia and patella.

3. The method of claim 1 where the joint is an ankle and the anatomical structures are the tibia, fibula and talus.

4. The method of claim 1 where the joint is an elbow and the anatomical structures are the ulna, a humerus and a radius.

5. The method of claim 2 that includes the step of locating a patella of the knee joint using the surgical navigation system and wherein the biomechanical properties of the knee joint include the biomechanical properties of the patella.

6. The method of claim 5, wherein the locating of the patella is done with at least three degrees of freedom.

7. The method of claim 6 that includes the step of choosing a patella implant based on interaction between the biomechanical properties of the femur and the biomechanical properties of the patella and on an attachment relationship of the patella to the tibia.

8. The method of claim 2, wherein the biomechanical properties include at least one of the location of the head of the femur, the digitization of the anterior cortex of the femur, the digitization of the center of the tibia, the digitization of the plateau dishes of the tibia, the location of the center of the ankle, and an initial functional flexion axis of the knee.

9. The method of claim 8, wherein the biomechanical properties also include a derived property based on the determined biomechanical properties.

10. The method of claim 9 wherein the derived properties include the surface of the femoral condyles, the center of the knee joint, the internal/external rotation of the femur, the internal/external rotation of the tibia, and the momentary rotation axes of the knee.

11. The method of claim 10 wherein the external/internal rotation of at least one of the femur, and the tibia is determined by flexing the knee.

12. The method of claim 10 wherein the external/internal rotation or the medial/lateral displacement of the femur is based on a patella trajectory while flexing the knee.

13. The method of claim 10 wherein the external/internal rotation of the femur is determined using the shape of the posterior condyles of the femur.

14. The method of claim 1 wherein the evaluation of the soft tissue properties is done by applying stress loads to the joint, and by moving the joint throughout its range of motion.

15. The method of claim 14 wherein the joint is a knee and the evaluation of the soft tissue properties is done by applying valgus/varus loads to the knee, and by flexing the knee through the range of motion of the knee.

16. The method of claim 1, wherein the evaluation of the soft tissue properties is performed using a distracting device and moving the joint throughout its range of motion.

17. The method of claim 16, wherein the joint is the knee and evaluation of the soft tissue properties is performed using a distracting device and flexing the joint throughout its range of motion.

18. The method of claim 17 that includes the step of iteratively establishing a flexion axis during the evaluation of the soft tissue envelope.

19. The method of claim 17, wherein the tibia is prepared prior to evaluating the soft tissue envelope.

20. The method of claim 16, wherein the balancing of the soft tissues and positioning and sizing of the implant components take into consideration a load distribution on the bearing surfaces of the components throughout the range of motion of the joint.

21. The method of claim 20, wherein the load distribution is assessed with sensing elements embedded in the distraction device.

22. The method of claim 2, wherein the manipulation of the soft tissue envelope properties, the biomechanical properties, and the chosen implants restore the knee to a computed flexion axis.

23. The method of claim 22 wherein the neutral flexion axis of the knee is calculated based on the varus/valgus angle throughout a range of flexion.

24. The method of claim 2, wherein the manipulation of the soft tissue envelope properties, and the chosen implants respect biomechanical properties of the unaffected tibio-femoral joint, patello-femoral joint or a blend between the two.

25. The method of claim 24, wherein the manipulation of the soft tissue envelope properties, and the chosen implants restore the knee to the unaffected joint line.

26. The method of claim 24, wherein the manipulation of the soft tissue envelope properties, and the chosen implants restore the knee to the unaffected joint line and the joint line is derived from an attachment relationship of the patella to the tibia.

27. The method of claim 24, wherein the manipulation of the soft tissue envelope properties, and the chosen implants restore the knee to aspects of bony landmarks.

28. The method of claim 24, wherein the manipulation of the soft tissue envelope properties, and the chosen implants restore the knee to a blend of the properties of the unaffected joint line and aspects of bony landmarks.

29. The method of claim 1, wherein the interactive view includes a preview of the chosen implants placed in proposed resections of the bones.

30. The method of claim 1, wherein the optimized position of the implant components takes into account the kinematical soft tissue constraints and those intrinsic to the prosthetic system being used.

31. The method of claim 30, wherein the optimization strategy can be a blend or biased interactively towards the soft tissue constraints or towards performance of the implant.

32. The method of claim 29, wherein the interactive view also includes a display of a gap between the articulation surfaces of the implant at any position of the joint.

33. The method of claim 29, wherein the joint is a knee and the interactive view also includes a display of a gap between the femoral implant and the tibial implant at any flexion of the knee joint.

34. The method of claim 1, wherein the interactive view includes multiple views of the joint.

35. The method of claim 1, wherein the implant is chosen from a database of implants.

36. The method of claim 35, wherein the choice of implant takes into consideration gender and racial characteristics.

37. The method of claim 1 wherein the display shows a sizing grid to assist in the choice of implant components.

38. The method of claim 2 wherein the interactive view also displays the gap between the femur and the tibia.

39. The method of claim 1 wherein the information is displayed with augmented reality techniques directly in situ.

40. The method of claim 39 wherein the information displayed as a model of the implant components superimposed at the correct position on the anatomical structures of the joint.

41. The method of claim 39 wherein the information displayed shows a required preparation of the joint according to the position of the implant components used.

42. The method of claim 41 wherein the information displayed also includes the necessary instrumentation to perform the preparation and where the instrumentation is shown at the correct position relative to the anatomical structures of the joint.

43. The method of claim 41 wherein the information displayed also includes the preparation boundaries and envelopes required by the instrumentation to perform the preparation and where the boundaries and envelopes are shown at the correct position relative to the anatomical structures of the joint.

44. The method of claim 1, wherein the surgeon balances the gap at any joint position.

45. The method of claim 44, wherein the joint is the knee and the surgeon balances the gap at any joint position or throughout flexion and extension.

46. The method of claim 2, wherein the balancing takes into consideration the medial-lateral discrepancy of the joint gap due to rotation of the tibia while flexing the joint.

47. The method of claim 1, wherein preoperatively measured biomechanical properties are taken into account.

48. The method of claim 47, wherein the measurement consists of motion analysis of the patient's limb while performing activities relevant to the patient's life style.

49. The method of claim 47, wherein the measurement consists of neuro-muscular activity of the patient's limb while performing activities relevant to the patient's life style.

50. The method of claim 47, wherein the preoperatively measured biomechanical properties can be compared with those measured after the procedure.

51. The method of claim 1, where the knowledge of the implant components allows for a reduced set of instrumentation to solely those required for implantation of said components.

52. The method of claim 1, where the preparation of the bones to receive the implants is done with the aid of motion constraining devices.

53. The method of claim 52, where the motion constraining device is a passive manipulator.

54. The method of claim 53, where the motion constraining device is an in-situ mounted manipulator.

55. The method of claim 52, where the motion constraining device is an active manipulator.

56. The method of claim 52, where the motion constraining device is a tele-manipulator.
